(19) United States
(12) Patent Application Publication
(10) Pub. No.: US 2025/0169941 A1
(43) Pub. Date: May 29, 2025

(54) PROSTHESIS FOR ATRAUMATICALLY GRASPING INTRALUMENAL TISSUE

(71) Applicant: Edwards Lifesciences CardiAQ LLC, Irvine, CA (US)

(72) Inventors: Jeremy Brent Ratz, Winchester, MA (US); Arshad Quadri, West Hartford, CT (US); Luca Pesce, Huntington Beach, CA (US)

(21) Appl. No.: 19/025,780

(22) Filed: Jan. 16, 2025

Related U.S. Application Data
(63) Continuation of application No. 17/733,914, filed on Apr. 29, 2022.
Application No. 17/733,914 is a continuation of application No. 16/783,868, filed on Feb. 6, 2020, now Pat. No. 11,324,591.
Application No. 16/783,868 is a continuation of application No. 15/947,168, filed on Apr. 6, 2018, now Pat. No. 10,583,000.
Application No. 15/947,168 is a continuation of application No. 14/197,690, filed on Mar. 5, 2014, now abandoned.
(60) Provisional application No. 61/782,707, filed on Mar. 14, 2013.

(51) Int. Cl. A61F 2/24

(52) U.S. Cl. CPC A61F 2/2409; A61F 2/2418; A61F 2/24; A61F 2/2457; A61F 2220/0008

(57) ABSTRACT

A prosthesis can comprise an expandable frame, a plurality of distal anchors and a plurality of proximal anchors. The anchors can extend outwardly from the frame. The frame can be configured to radially expand and contract for deployment within a body cavity. The frame and anchors can have one of many different shapes and configurations. For example, the anchors can have looped ends, and the entire anchor may loop out from the frame.

### CROSS-REFERENCE TO RELATED APPLICATIONS

This application is a continuation of U.S. patent application Ser. No. 17/733,914, filed Apr. 29, 2022, which is a continuation of U.S. patent application Ser. No. 16/783,868, filed Feb. 6, 2020, now U.S. Pat. No. 11,324,591, which is a continuation of U.S. patent application Ser. No. 15/947,168, filed Apr. 6, 2018, now U.S. Pat. No. 10,583,000, which is a continuation of U.S. patent application Ser. No. 14/197,690, filed Mar. 5,2014, which claims the benefit of priority to U.S. Provisional Appl. No. 61/782,707, filed Mar. 14, 2013. The entire contents of the above applications are hereby incorporated by reference and made a part of this specification.

### BACKGROUND OF THE INVENTION

### Field of the Invention

Certain embodiments disclosed herein relate generally to prostheses for implantation within a lumen or body cavity. In particular, certain embodiments relate to expandable prostheses such as replacement heart valves, such as for the mitral valve, that are configured to atraumatically grasp intralumenal tissue.

### Description of the Related Art

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner. Further challenges arise when trying to controllably deliver and secure such prostheses in a location such as at a native mitral valve.

### SUMMARY OF THE INVENTION

Embodiments of the present disclosure are directed to a prosthesis, such as but not limited to a replacement heart valve. Further embodiments are directed to methods of delivering a prosthesis into a body cavity and/or securing a prosthesis to intralumenal tissue. In some embodiments, a replacement heart valve and methods for delivering a replacement heart valve to a native heart valve, such as a mitral valve, are provided.

In some embodiments a prosthesis can comprise an expandable frame, a plurality of distal anchors and a plurality of proximal anchors. The anchors can extend outwardly from the frame. The frame can be configured to radially expand and contract for deployment within the body cavity. In some embodiments, when the frame is in an expanded configuration, the frame can have a larger cross-sectional dimension in a middle portion of the frame and a smaller cross-sectional dimension in a proximal portion and a distal portion of the frame, wherein the middle portion is between the proximal and distal portions. In some embodiments, at least some of the anchors comprise a loop that forms an atraumatic end of a corresponding anchor.

In some embodiments, a prosthesis can be configured to atraumatically grasp intralumenal tissue when deployed within a body cavity. The prosthesis can comprise an expandable frame comprising a proximal end and a distal end and having a longitudinal axis extending between the proximal end and the distal end. The frame can be configured to radially expand and contract for deployment within the body cavity, wherein when the frame is in an expanded configuration, the frame has a larger cross-sectional dimension in a middle portion of the frame and a smaller cross-sectional dimension in a proximal portion and a distal portion of the frame, wherein the middle portion is between the proximal and distal portions. The prosthesis can also include a plurality of generally distally extending anchors extending from the proximal portion of the frame and configured so that when the frame is in an expanded configuration each distally extending anchor has an end positioned radially outward from the middle portion of the frame, and a plurality of generally proximally extending anchors extending from the distal portion of the frame and configured so that when the frame is in an expanded configuration each proximally extending anchor has an end positioned radially outward form the middle portion of the frame and axially spaced from the ends of the distally extending anchors. At least some of the anchors can comprise a loop that forms an atraumatic end of the anchor The frame can be configured such that radial expansion of the frame causes the ends of the plurality of distally extending anchors and the ends of the plurality of proximally extending anchors to draw closer together.

A prosthesis according to certain embodiments can be configured to atraumatically grasp intralumenal tissue when deployed within a body cavity. The prosthesis can comprise an expandable frame, a plurality of proximal anchors each connected to the frame so that when the frame is in an expanded configuration an end of each proximal anchor is positioned radially outward from the frame and extends generally distally, and a plurality of distal anchors each connected to the frame so that when the frame is in an expanded configuration an end of each distal anchor is positioned radially outward from the frame and extends generally proximally, wherein the ends of the distal anchors are axially spaced from the ends of the proximal anchors when the frame is in an expanded configuration. In some embodiments, at least some of the anchors can comprise a looped end.

Alternatively, or in addition, in some embodiments, at least some of the anchors can comprise a loop. Each loop of these later embodiments can comprise first through fourth segments and an arcuate segment. The first and second segments can both extend in a first generally axial direction away from the frame. The third and fourth segments can extend radially outward from the frame in a second direction generally opposite the first direction, the third segment connected to the first segment and the fourth segment connected to the second segment. The arcuate segment can connect the third segment and the fourth segment that forms an atraumatic end of a corresponding anchor. In some embodiments, the frame is configured such that radial expansion of the frame causes the ends of the plurality of proximal anchors and the ends of the plurality of distal anchors to draw closer together.

In some embodiments, a prosthesis can be configured to atraumatically grasp intralumenal tissue when deployed within a body cavity. The prosthesis can comprise an expandable frame, a plurality of proximal anchors, and a plurality of distal anchors. The expandable frame can comprise a proximal end and a distal end and having a longitudinal axis extending between the proximal end and the distal end, the frame configured to radially expand and contract for deployment within the body cavity. The plurality of proximal anchors can each connect to the frame so that when the frame is in an expanded configuration an end of each proximal anchor is positioned radially outward from the frame and extends generally distally. The plurality of distal anchors can each connect to the frame so that when the frame is in an expanded configuration an end of each distal anchor is positioned radially outward from the frame and extends generally proximally, wherein the ends of the distal anchors are axially spaced from the ends of the proximal anchors when the frame is in an expanded configuration. At least some of the anchors can comprise a loop. Each of the anchors that comprises a loop can comprise at least a first segment extending in a first generally axial direction away from the frame, and a second segment and a third segment extending radially outward from the frame in a second direction generally opposite the first direction and coming together in an atraumatic end. The frame can be configured such that radial expansion of the frame causes the ends of the plurality of proximal anchors and the ends of the plurality of distal anchors to draw closer together.

According to some embodiments a prosthesis can be configured to atraumatically grasp intralumenal tissue when deployed within a body cavity. The prosthesis can comprise an expandable frame comprising a proximal end and a distal end and having a longitudinal axis extending between the proximal end and the distal end, the frame configured to radially expand and contract for deployment within the body cavity, a plurality of proximal anchors each connected to the frame so that when the frame is in an expanded configuration an end of each proximal anchor is positioned radially outward from the frame and extends generally distally, and a plurality of distal anchors each connected to the frame so that when the frame is in an expanded configuration an end of each distal anchor is positioned radially outward from the frame and extends generally proximally, wherein the ends of the distal anchors are axially spaced from the ends of the proximal anchors when the frame is in an expanded configuration. At least some of the anchors can comprise a loop and each loop can comprise first through fourth segments and an arcuate segment. The first and second segments can both extend in a first generally axial direction away from the frame. The third and fourth segments can extend radially outward from the frame in a second direction generally opposite the first direction, the third segment connected to the first segment and the fourth segment connected to the second segment. The arcuate segment can connect the third segment and the fourth segment that forms an atraumatic end of a corresponding anchor. The frame can be configured such that radial expansion of the frame causes the ends of the plurality of proximal anchors and the ends of the plurality of distal anchors to draw closer together.

Methods of delivering a prosthesis and/or securing the prosthesis to intralumenal tissue are also provided. In one embodiment, a method of delivering a replacement valve to a native mitral valve can comprise one or more of the following steps. Delivering a replacement valve mounted on a delivery device to the native mitral valve annulus while the replacement valve is in a radially compacted state, the replacement valve comprising a radially expandable frame comprising a proximal end, a distal end, a plurality of distal anchors extending generally proximally from the frame, and a plurality of proximal anchors extending generally distally from the frame. Positioning the replacement valve so that ends of the distal anchors are on a ventricular side of the native leaflets beyond a location where chordae tendineae connect to free ends of the native leaflets. Releasing at least a portion of the replacement valve from the delivery device to thereby expand the distal anchors radially outwardly to a first radial dimension. Moving the ends of the distal anchors toward the ventricular side of the native valve annulus with the distal anchors extending between at least some of the chordae tendineae to provide tension on the chordae tendineae. Further releasing the replacement valve from the delivery device to thereby expand the proximal anchors radially outwardly to a second radial dimension greater than the first radial dimension, wherein the proximal anchors upon further release of the replacement valve from the delivery device move into engagement with tissue on an atrial side of the native valve annulus while the distal anchors provide tension on the chordae tendineae.

### BRIEF DESCRIPTION OF THE DRAWINGS

These and other features, aspects and advantages are described below with reference to the drawings, which are intended to illustrate but not to limit the invention. In the drawings, like reference characters denote corresponding features consistently throughout similar embodiments.

FIG. 1A is a side view of an embodiment of a prosthesis.

FIG. 1B is a flat pattern view of the prosthesis of FIG. 1A.

FIG. 2A is a side view of an embodiment of a prosthesis.

FIG. 2B is a flat pattern view of the prosthesis of FIG. 2A.

FIG. 3A is a side view of an embodiment of a prosthesis.

FIG. 3B is a flat pattern view of the prosthesis of FIG. 3A.

FIG. 4 is a side view of an embodiment of a prosthesis.

FIG. 5 is a side view of an embodiment of a prosthesis.

FIG. 6A is a side view of an embodiment of a prosthesis configured as a replacement heart valve.

FIG. 6B is a bottom view of the prosthesis similar of FIG. 6A.

FIG. 7 is a side view of an embodiment of a prosthesis configured as a replacement heart valve.

FIG. 8A is a side view of an embodiment of a prosthesis.

FIG. 8B is a detail view of a portion of the prosthesis of FIG. 8A.

FIG. 9A is a side view of an embodiment of a prosthesis.

FIG. 9B is a bottom view of the prosthesis of FIG. 9A, configured as a replacement heart valve.

FIG. 10 is a schematic representation of a prosthesis positioned within the heart.

FIG. 10A is a detail schematic representation of the prosthesis positioned within the heart of FIG. 10.

### DETAILED DESCRIPTION OF THE PREFERRED EMBODIMENTS

The present specification and drawings provide aspects and features of the disclosure in the context of several embodiments of prostheses, replacement heart valves, delivery devices and methods that are configured for use in the vasculature of a patient, such as for replacement of natural heart valves in a patient. These embodiments may be discussed in connection with replacing specific valves such as the patient's aortic or mitral valve. However, it is to be understood that the features and concepts discussed herein can be applied to products other than heart valve implants. For example, the controlled positioning, deployment, and securing features described herein can be applied to medical implants, for example other types of expandable prostheses, for use elsewhere in the body, such as within a vein, or the like. In addition, particular features of a valve, delivery device, etc. should not be taken as limiting, and features of any one embodiment discussed herein can be combined with features of other embodiments as desired and when appropriate.

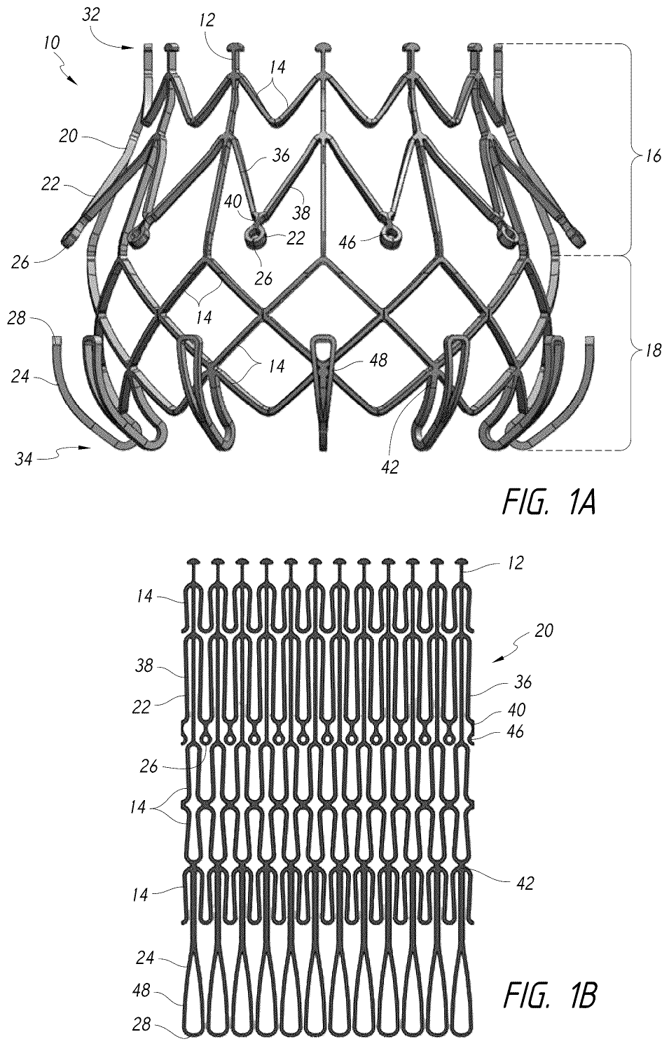

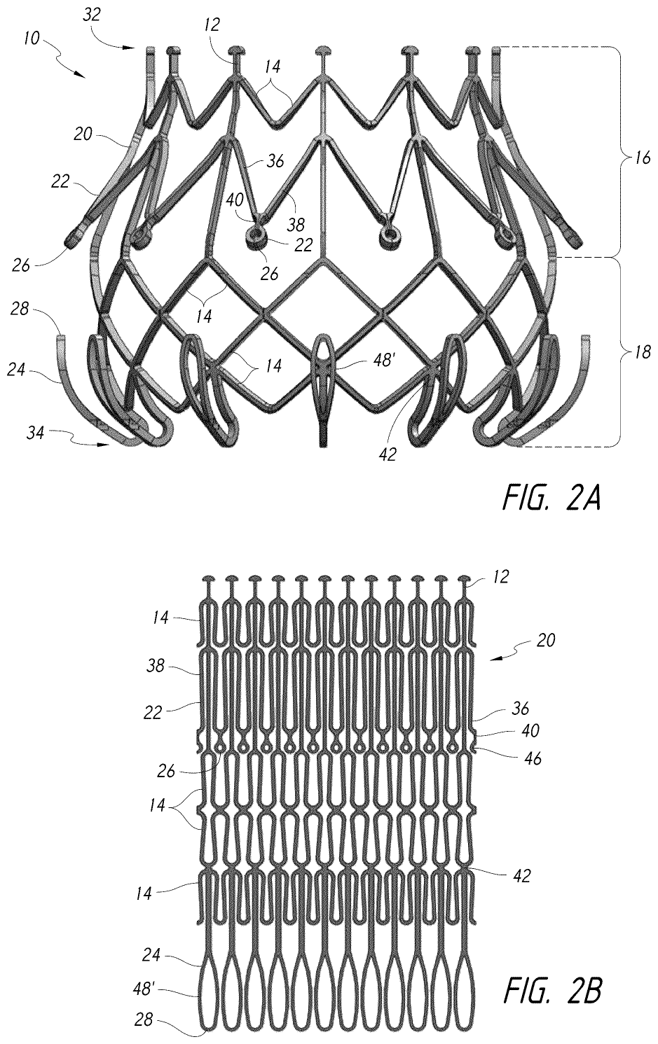

With initial reference to FIG. 1A, an embodiment of a prosthesis 10 is shown. The illustrated prosthesis 10 includes a frame 20 that may be self-expanding or balloon expandable. The prosthesis may further include a replacement valve that can be designed to replace a damaged or diseased native heart valve such as a mitral valve. The replacement valve is not shown in this embodiment as to more clearly illustrate features of the frame 20, though it will be understood that a replacement valve is not required as part of the prosthesis. In addition, it will be understood that only a front portion of the frame 20 is shown for further ease of illustration.

The frame 20 can be made of many different materials, but is preferably made from metal. In some embodiments, the frame 20 can be made from a shape memory material, such as nitinol. A wire frame or a metal tube can be used to make the frame. The wire frame of a metal tube can be cut or etched to remove all but the desired metal skeleton. In some embodiments a metal tube is laser cut in a repeating pattern to form the frame. FIG. 1B illustrates the flat cut pattern of the frame shown in FIG. 1A. The flat pattern can be cut from a metal tube and then the tube can be bent and expanded to the shape shown in FIG. 1A. The frame 20 can further be expanded and/or compressed and/or otherwise worked to have the desired shape or shapes, such as for introduction and implantation.

As shown, the frame when in an expanded configuration, such as in a fully expanded configuration, has a bulbous or slightly bulbous shape, with a middle portion being larger than the proximal 32 and distal 34 ends. In some embodiments, the inside diameter of the both ends can be the same, or it can be bigger on one end than the other, while still having a middle portion larger than both the proximal and distal ends. In some embodiments, the effective diameter of the distal frame end is smaller than the effective diameter of the middle portion. The bulbous shape of the frame can advantageously allow the frame to engage a native valve annulus or other body cavity, while spacing the inlet and outlet from the heart or vessel wall. This can help reduce undesired contact between the prosthesis and the heart or vessel, such as the ventricular wall of the heart. In other embodiments, the frame may not have a bulbous portion, and can have substantially the same outer dimension along its entire length, or it may have one end larger than the other end. The prosthesis 10 and frame 20 may be similar to the replacement heart valves and associated frames disclosed in U.S. Pat. No. 8,403,983 and U.S. Publication Nos. 2010/0298931, 2011/0313515 and 2012/0078353 the entireties of each of which are hereby incorporated by reference and made a part of this specification. This is inclusive of the entire disclosure and is not in any way limited to the disclosure of the replacement heart valves and associated frames.

A number of struts collectively make up the frame 20. FIG. 1 illustrates the frame in an expanded configuration with a number of longitudinal struts 12 and undulating struts 14, with cells defined by the open spaces between the struts. The longitudinal struts may be arranged so that they are parallel or generally or substantially parallel to a longitudinal axis of the frame. The longitudinal axis of the frame may be defined as the central axis that extends through the center of the frame between the proximal 32 and distal 34 ends. Any number of configurations of struts can be used, such as the rings of undulating struts shown forming chevrons and diamonds, but also ovals, curves, and various other shapes. The illustrated embodiment includes two rings, or rows of chevrons shown in portion 16 and two rows of diamond-shaped cells shown in portion 18.

The frame 20 has a non-foreshortening portion 16 and a foreshortening portion 18. These portions can be defined by the frame 20 and the positioning of various types of struts along the frame 20. In FIG. 1 it can be seen that the longitudinal struts 12 span the length of the non-foreshortening portion 16, while undulating struts 14 form the foreshortening portion 18. When the frame is radially collapsed or compacted, the struts 14 become more parallel with respect to the longitudinal axis of the frame, causing an outer diameter of the frame to decrease and the longitudinal length of the frame to increase in the foreshortening portion 18. As the frame moves from a compacted position to an expanded position, the longitudinal length of the frame can decrease in the foreshortening portion 18. But, the frame length does not substantially change length in the non-foreshortening portion 16.

Foreshortening of the frame 20 can be used to engage and secure the prosthesis to intralumenal tissue in a body cavity, for example tissue at or adjacent a native valve, such as a native valve annulus and/or leaflets. Opposing anchors 22, 24 can be constructed on the frame 20 so that portions of the anchors, such as tips or ends 26, 28, move closer together as the frame foreshortens. As one example, this can allow the anchors 22, 24 to grasp tissue on opposite sides of the native mitral annulus to thereby secure the prosthesis at the mitral valve.

The anchors 22, 24 and anchor tips 26, 28 can be located anywhere along the frame 20 just so long as at least one of the anchors is either connected to the foreshortening portion 18 or the foreshortening portion is positioned between the anchors so that a portion of the anchors will be move closer together with expansion of the frame. As shown, the anchors 24 are connected to the foreshortening portion 18. The foreshortening portion can also be positioned anywhere along the frame, though it is shown towards the distal end 34. In some embodiments, both of the anchor tips 26, 28 are located in the foreshortening portion 18. In some embodiments, the foreshortening portion 18 may extend the entire length of the frame, such that there is no non-foreshortening portion 16.

Preferably, each of the anchors 22, 24 is positioned or extends generally radially outwardly from the frame 20 so that the anchor tips 26, 28 are generally spaced away or radially outward from the rest of the frame 20. For example, the anchor tips may be located radially outward from the middle portion of the frame, with the tips 26 and 28 being axially spaced from one another. In some embodiments, all or part of the structure connected to the anchor tip and extending radially from the frame, including one or more rings and/or struts, can be considered part of the anchor. The anchors can include a base located on the anchor on a side opposite the tip. The base can be for example where the anchor begins to extend from or away from the frame 20.

For example, proximal anchors 22 are shown having first 36 and second 38 struts forming a chevron and connected to longitudinal struts 12 at a base of the anchor. The first and second struts of the anchor 22 are bent at the base so that the anchor 22 extends radially outwardly from the frame as it extends generally distally towards the tip 26. The first and second struts can be connected to each other at a radially outward location to form an outwardly extending loop, and in some embodiments, the first and second struts can be joined at a third strut 40 that continues to extend outwardly and/or generally distally. Here the third strut 40 is a short strut. The anchor also includes an eyelet 46. As illustrated, the eyelet is located at the distal end 26, though the eyelet can be positioned in other locations along the anchor 22. The tips 26 of the proximal anchors may extend distally and be parallel or substantially parallel with the longitudinal axis of the frame, or as illustrated in FIG. 1A, the tips 26 may extend generally distally but still radially outwardly inclined or at an acute angle relative to the longitudinal axis of the frame.

As another example, the distal anchors 24 are shown having looped ends 48. The looped ends can be larger near the tip to form a type of elongated teardrop. In addition, the tips 28 may be substantially flat. The looped end may assist the frame in not getting caught up on structures at or near the treatment location. For example, each loop can be configured so that when the frame is deployed in-situ and expands, the movement of each loop from a delivered position to a deployed position can avoids getting caught on the papillary muscles.

Each distal anchor 24 is connected to the frame at a base 42. As illustrated in FIG. 1A, the base of the distal anchor may be at a location where the corners of adjacent cells meet, such that the base is proximal to the distal end 34 of the frame. In other embodiments, the base of the distal anchor may be at a distal most corner of a cell, which corresponds to a distal most point on the frame The distal anchors as illustrated extend from the base 42 generally distally before bending back around in an arcuate segment where the distal anchor extends generally proximally and radially outwardly from the frame. As shown, the anchors 24 may also generally distally and radially inwardly with respect to the frame such that the distal most point on the prosthesis has a smaller inside diameter than where the base 42 connects to the frame. The inside diameter at the distal most can be the same or substantially the same as the inside diameter of the proximal end, or may be smaller. The anchor as illustrated is bent around about 180 degrees so that the tip 28 extends in the opposite, proximal direction, which may be parallel or substantially parallel to the longitudinal axis of the frame. For example, in FIG. 1A it can be seen that the distal anchors 24 are bent further inward such that the ends of the anchors point proximally and are generally parallel with the longitudinal axis of the frame. Alternatively, the tip 28 may extend generally proximally but still extend radially outwardly inclined or at an acute angle relative to the longitudinal axis of the frame

It will be understood that the anchors can have various other configurations, including the various embodiments that follow. In some embodiments, each of the anchors can extend radially outwardly from the frame at an anchor base and terminate at an anchor tip. The anchors can be connected to the frame at one of many different locations including apices, junctions, other parts of struts, etc. The anchors can comprise first, second, third, or more spaced apart bending stages along the length of each anchor. The anchors can also extend either distally or proximally before and/or after one or more of the bending stages. A portion of the anchor may extend with the frame before or after any bending stages.

In the illustrated embodiment of FIG. 1A-B there are twelve distal anchors and twelve proximal anchors. In some embodiments there may be 6 anchors on one side and 12 on the other. Some embodiments may include different numbers of anchors. In addition, the distal and proximal anchors may be aligned so the tips point generally towards each other, or they may be spaced so that the tips point between two tips on the opposite side, as is illustrated in FIGS. 1A-B.

The anchor tips 26 and 28 as described above advantageously provide atraumatic surfaces that may be used to grasp intralumenal tissue without causing unnecessary or undesired trauma to tissue. For example, the proximal anchors tips 26 and distal anchor tips 28 may form flat, substantially flat, curved or other non-sharp surfaces to allow the tips to engage and/or grasp tissue, without necessarily piercing or puncturing through tissue.

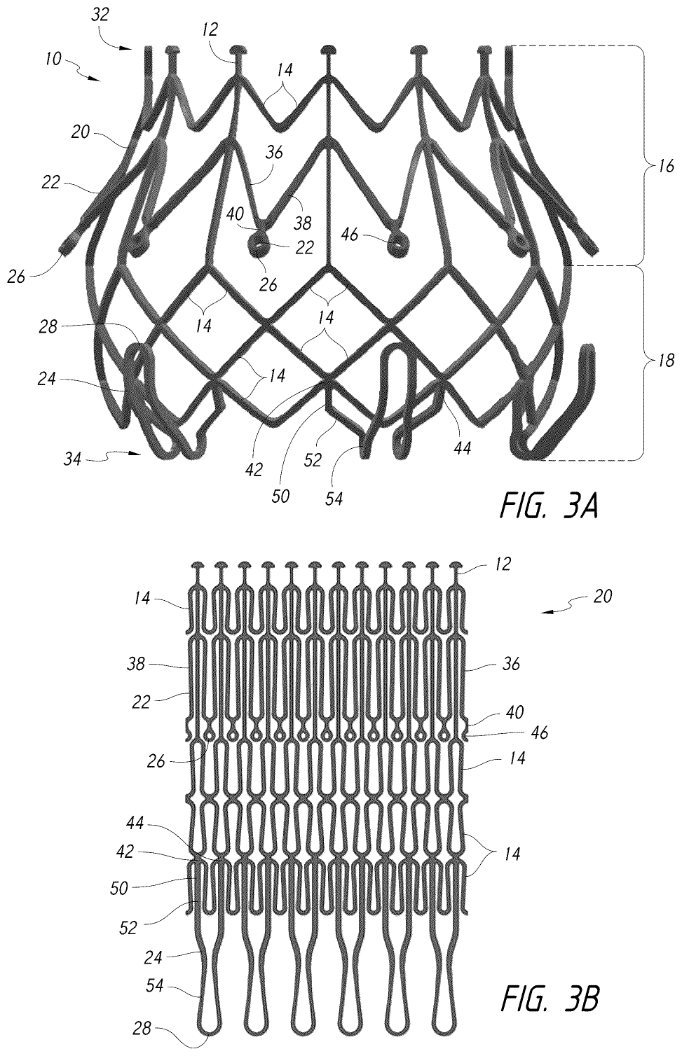

FIGS. 2A-3B show prostheses similar to that of FIGS. 1A-B with two different styles of distal anchors 24. In FIGS. 2A-B, the looped end 48′ of the distal anchor is generally more elliptical with a curved tip as compared to the elongated teardrop shape of looped end 48 of FIGS. 1A-B. Otherwise the shape is substantially the same.

In FIGS. 3A-B, the distal anchors 24 are looped anchors rather than having looped ends. The looped anchor has a first base 42 and a second base 44 connected to the frame, wherein the first and second bases are at opposite corners of the same cell. Alternatively, the first and second bases may be located at the distal most corners of adjacent cells. The distal anchors 24 extends generally distally from the frame at the first base 42 but then is bent back around and begins to extend outwardly from the frame in a generally proximal direction. The distal anchor 24 then repeats this configuration in reverse towards the second base 44 such that the two sides of the looped anchor are mirror images of one another. It will be understood that the looped anchor can have other configurations and that it may not be symmetrical.

As illustrated in FIG. 3A, the tips 28 of the distal anchors are circumferentially aligned with the tips 26 of the proximal anchors, though in other embodiments, the tips 28 of the distal anchors may be circumferentially staggered between the tips 26 of the proximal anchors. In the embodiment of FIG. 3A, adjacent distal anchors 26 are spaced apart by one cell, though in other embodiments, adjacent distal anchors may be provided on adjacent cells. Thus, for example, instead of having six distal anchors and twelve proximal anchors as shown in FIG. 3A, there may be a 1:1 correspondence between proximal and distal anchors.

The illustrated looped distal anchor of FIGS. 3A-B is made up of the following segments. The first segment 50 extends generally longitudinally with the frame, extending distally or generally distally (e.g., slightly radially inward) with the frame. The strut is then bent so that a second segment 52 extends generally parallel with an adjacent undulating strut 14. The strut is then bent so that a third segment 54 begins to extend generally longitudinally and distally or generally distally, and then is bent back around to point in generally the opposite direction (e.g., in a proximal direction parallel or generally parallel with the longitudinal axis of the frame). The third segment 54 ends in the rounded tip 28 and then the anchor strut repeats to form the mirror image. After the third segment 54 bends back around to point in generally the opposite direction, in the embodiment illustrated the third segment may first extend radially outward at an acute angle relative to the longitudinal axis before bending into a portion that extends parallel or substantially parallel to the longitudinal axis. The paired third segments 54 extend parallel or generally parallel with one another from the second segment to the tip, though they may also move slightly towards or away from each other in some embodiments.

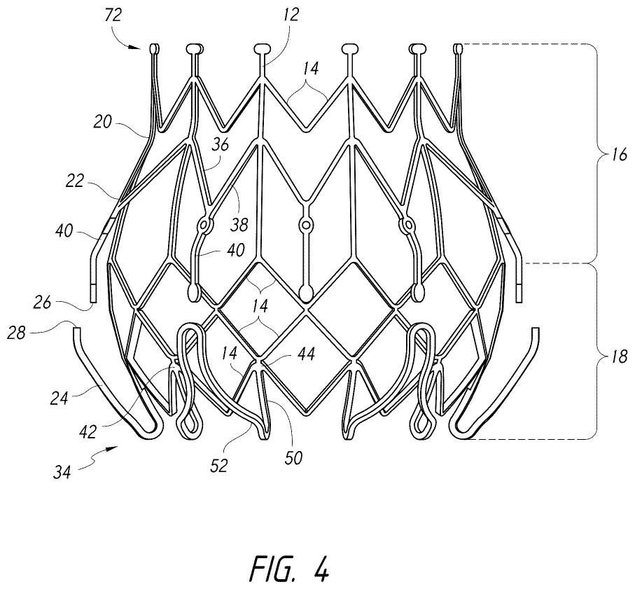

FIG. 4 shows a prosthesis similar to FIGS. 3A-B that also has looped distal anchors. In this embodiment the first segment 50 extends longitudinally in a distal direction from the frame and the strut is bent back on itself to point generally in the opposite (e.g., proximal) direction. The second segment is bent inward before extending parallel or generally parallel with its mirror image on the other side forming a nose and wing configuration similar to the shape of certain bicycle seats.

The proximal anchors 22 also have an elongated third strut 40. The proximal anchor 22 is shown having first 36 and second 38 struts forming a chevron and connected to longitudinal struts 12 at a base of the anchor. The first and second struts of the anchor 22 are bent at the base so that the anchor 22 extends radially outwardly from the frame as it extends towards the tip 26. The first and second struts join at a third strut 40 that continues to extend outwardly and is then bent such that the tip points distally and extends in a manner parallel or generally parallel with the longitudinal axis of the frame. The proximal anchor may or may not include an eyelet 46 along its length. The distal tip of the proximal anchors may have an atraumatic surface, such as an enlarged circular or curved end as illustrated. When the frame is in an expanded configuration, the distal anchors 24 may have tips 28 that are positioned radially outward of the tips 26 of the proximal anchors 22. Other embodiments may have the tips 26 being positioned outward of the tips 28. Such configurations are also possible with the other frames and prostheses described elsewhere herein.

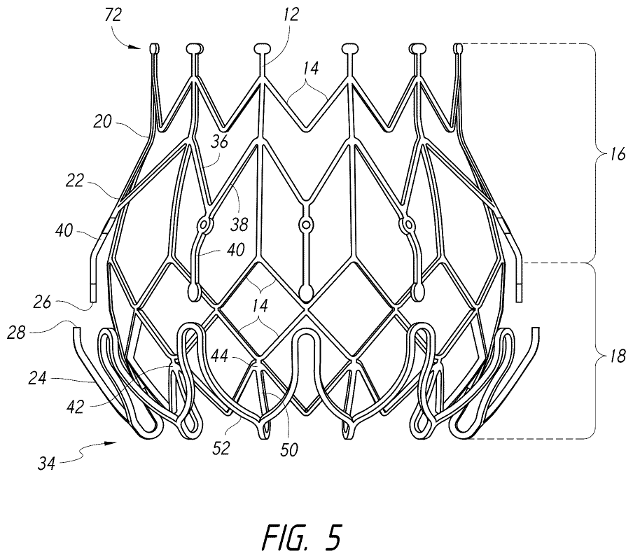

FIG. 5 illustrated an embodiment similar to the prosthesis of FIG. 4 with twelve distal anchors instead of six. Because of this change, in one embodiment two anchors share the first segment 50 where the anchor base 42, 44 is connected to the frame. As illustrated, each of the proximal and distal anchors may be circumferentially aligned with each other, and each of the distal anchors corresponds to one of the cells

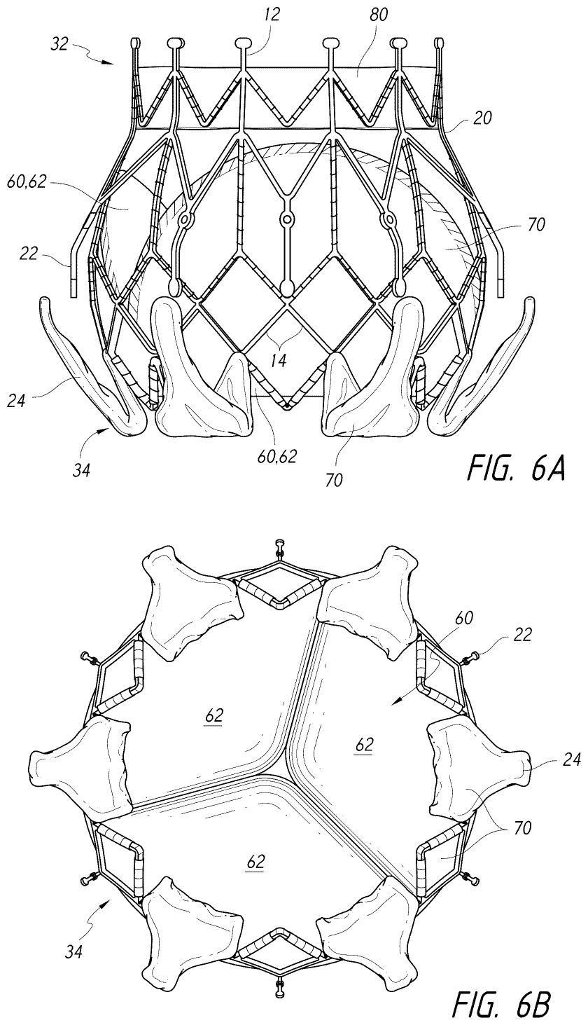

Turning now to FIGS. 6A-B, prosthesis and frame embodiments are shown similar to that of FIG. 4, including various other components of the prosthesis. A prosthesis can include one or more of a valve 60, a skirt 70 and a support band 80. The prosthesis can be a replacement heart valve similar to that and including features similar to those disclosed in U.S. patents application Ser. Nos. 13/165,721, filed Jun. 21, 2011, published as U.S. 2011/0313515; and 13/244,080, filed Sep. 23, 2011, published as 2012/0078353. The entire contents of both applications are hereby incorporated by reference herein and made a part of this specification. This is inclusive of the entire disclosure and is not in any way limited to the disclosure of the replacement heart valve.

The valve 60 can be a replacement heart valve which includes a plurality of valve leaflets 62. The plurality of valve leaflets 62 can function in a manner similar to the natural mitral valve, or to other valves in the vascular system. The plurality of valve leaflets 62 can open in a first position and then engage one another to close the valve in a second position. The plurality of valve leaflets 62 can be made to function as a one way valve such that flow in one direction opens the valve and flow in a second direction opposite the first direction closes the valve. The replacement heart valve 60 can be constructed so as to open naturally with the beating of the heart. For example, the plurality of valve leaflets 62 can open during diastole and close during systole.

In some embodiments, the leaflets 62 can be coupled to a skirt 70. For example, the proximal ends of the leaflets 62 can be connected to a proximal end of the skirt 70.

The skirt 70 can be used to at least partially control how fluid flows through and/or around the valve 60. The skirt 70 can surround at least a portion of the valve and be connected to the valve leaflets 62. In some embodiments, the skirt 70 can form an inner wall connected to and positioned within the frame 20. The skirt 70 can also be made to move with the foreshortening portion 18 of the frame 20.

The skirt 70 can extend the length of the frame 20 or it can extend along only part of the length of the frame 20. In some embodiments, the ends of the heart valve 60 can coincide with ends of the skirt 70. In addition, one or more of the ends of the frame 20 can coincide with the ends of the skirt 70. In the illustrated embodiment of FIGS. 6A-B, the proximal end of the skirt 70 and heart valve 60 are sewn together. The skirt 70 can not only extend to the distal end of the frame 20 but can also extend to the outside of the frame and is shown wrapped around each of the distal anchors 24.

Other shapes and configurations can also be used for the valve 60 and skirt 70. In some embodiments, the skirt 70 may extend along the length of the leaflets 62, but is not connected to them. In the illustrated embodiments, the skirt 70 is attached to the frame 20 and the leaflets 62 are attached to the skirt 70.

The skirt 70 can be constructed in multiple different ways. The skirt 70 can be made of knit polyester or another stretchable or flexible fabric. In some embodiments, the skirt 70 is made from a material that is more flexible than the valve leaflet material. The distal and/or proximal end of the skirt 70 can be straight, curved, or have any other desired configuration. For example, the skirt 70 is shown with undulations patterned to generally correspond to the undulations at the distal end 34 of the frame 20. It can be seen that the skirt 70 wraps around the struts at the distal end. The skirt 70 can be formed of one piece or multiple pieces. For example, the skirt 70 attached to the valve 60 can be one piece and then each distal anchor can be covered by a separate piece of material of the skirt 70. It is to be understood that other configurations of the skirt 70 can also be employed. For example, the anchors may remain uncovered, or only a portion may be covered.

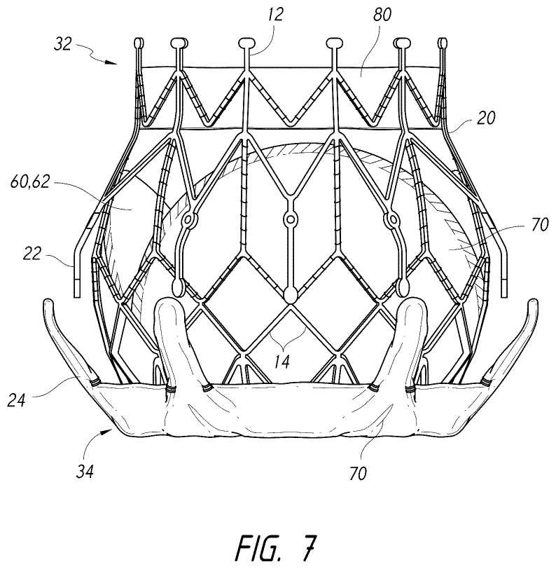

Turning now to FIG. 7, another embodiment of the skirt 70 is shown. Here rather than the skirt 70 corresponding to the undulations at the distal end 34 of the frame 20, the skirt extends past the frame and is then wrapped around it. Thus, the skirt 70 extends from the inside of the frame 20 to the outside of the frame. The skirt can extend completely around the frame for ¼, ⅓, ½, or more of the length of the distal anchors. The skirt can also cover the distal anchors 24. In the illustrated embodiment, the skirt is a one piece skirt, but it will be understood that the skirt can be made of multiple pieces.

The skirt 70, and particularly portions that cover the distal anchors 24, can beneficially be used to help prevent leakage of blood flow around the heart valve. In addition, the skirt can encourage tissue in-growth between the skirt and the natural tissue. This may further help to prevent leakage of blood flow around the heart valve.

The prosthesis 10 can also include a support band 80 as is shown in FIGS. 6A-7. The support band 80 may be placed or positioned around or within the frame 20 at the proximal end 32. The support band 80 can be used to reinforce and/or constrain the frame 20. The support band 80 can help to control the expansion of the frame 20 from the compacted to the expanded state. The support band 80 can also be used to reduce the amount of motion that occurs at the proximal end 32 after the prosthesis 10 has been implanted at the mitral heart valve or other location.

In some embodiments, the support band 80 may comprise a polyester fabric band. The support band 80 may comprise a no-stretch or limited stretch material. Preferably the support band 80 is not made of an elastic material or a material known to have high elasticity. In some embodiments, the support band 80 is made from a material that is less flexible than the valve skirt material and/or the valve leaflet material. The distal and proximal ends of the support band 80 can be straight, curved, undulating with the undulations of frame, or any other desired configuration.

The support band 80 can be connected to the valve frame with a plurality of stitches, loops, knots, staples, or other types of connections. In some embodiments, the frame 20 can be sandwiched between two sides or layers of the support band 80. Preferably, the support band 80 is a single layer positioned within and attached to the frame 20 with a plurality of stitches around one or more of the longitudinal and/or undulating struts. In some embodiments, the support band 80 can be attached to the proximal end of the valve skirt 40.

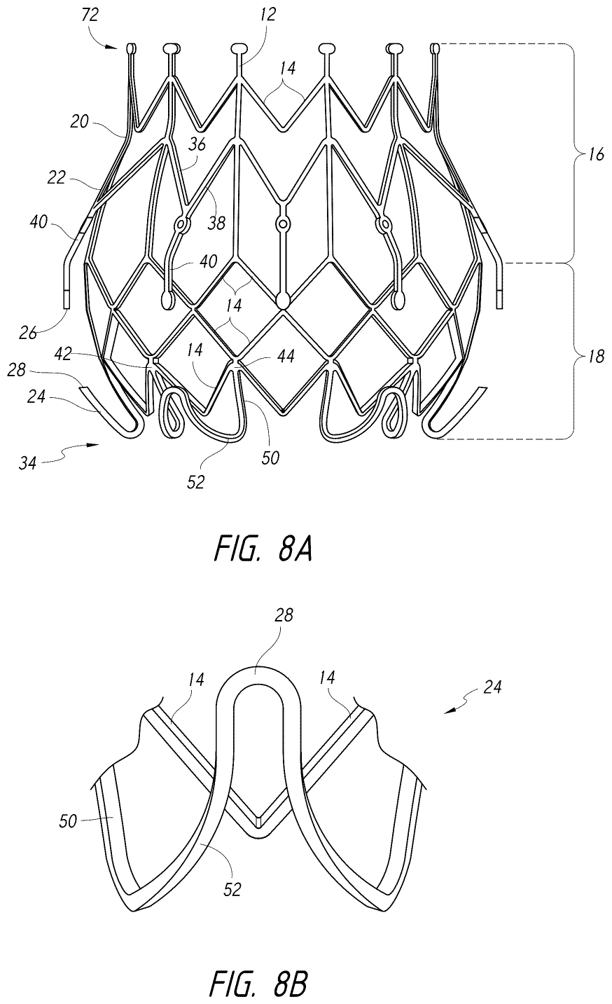

Looking now at FIGS. 8A-B another embodiment of a prosthesis 10 is shown. FIGS. 8A-B show a prosthesis similar to that of FIG. 4 with a different style and configuration of distal anchor 24. In FIGS. 8A-B, the distal anchors are shorter than and spaced radially inward from the distal anchors of FIG. 4. Thus, as illustrated, the distal anchors 24 are not positioned as far radially outward as the proximal anchors, and the tips 28 may be positioned radially inward of the tips 26. As described further below, such a configuration may be advantageous in positioning and securing the prosthesis in a mitral valve or other body location. As shown particularly in FIG. 8B, the distal anchors 24 may comprise loops as described above, having a curved or arcuate atraumatic tip to minimize damage to body tissue.

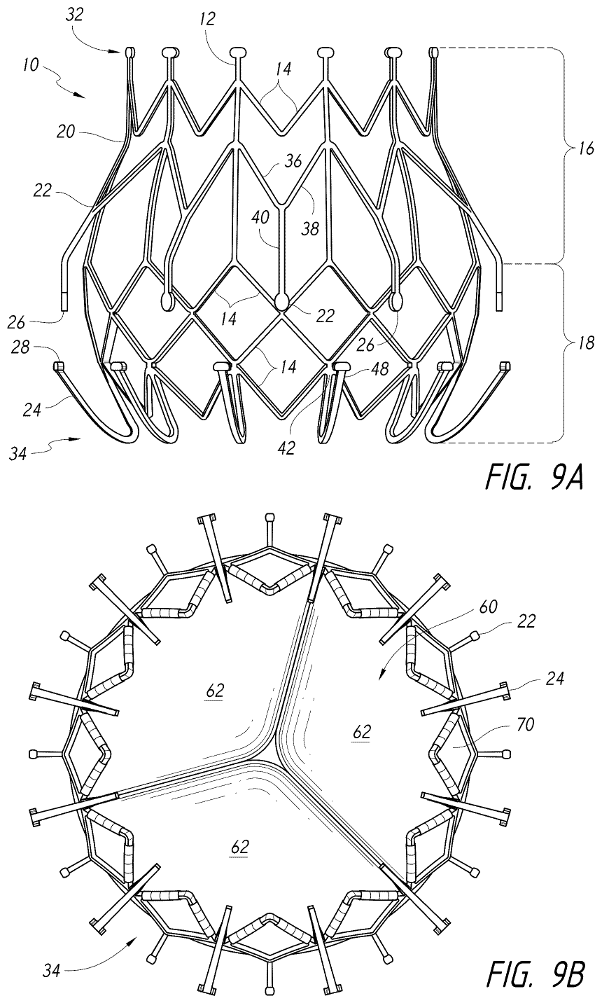

FIGS. 9A-B show an embodiment of a prosthesis where the distal anchors do not comprise loops, but instead comprise single struts each extending distally from the corners where adjacent cells meet. As described with respect to embodiments above, these anchors may first extend distally or generally distally, and may further extend radially inward, before bending around to extend proximally or generally proximally, such as at an acute angle relative to the longitudinal axis of the frame. The tips 28 of the anchors may comprise an atraumatic surface, such as a flattened or curved enlarged tip. As illustrated, the tips 28 may be circumferentially staggered between tips 26 of the proximal anchors 22, as best shown in FIG. 9B. FIG. 9B also shows the frame 20 having a valve 60 and skirt 70 attached as described above.

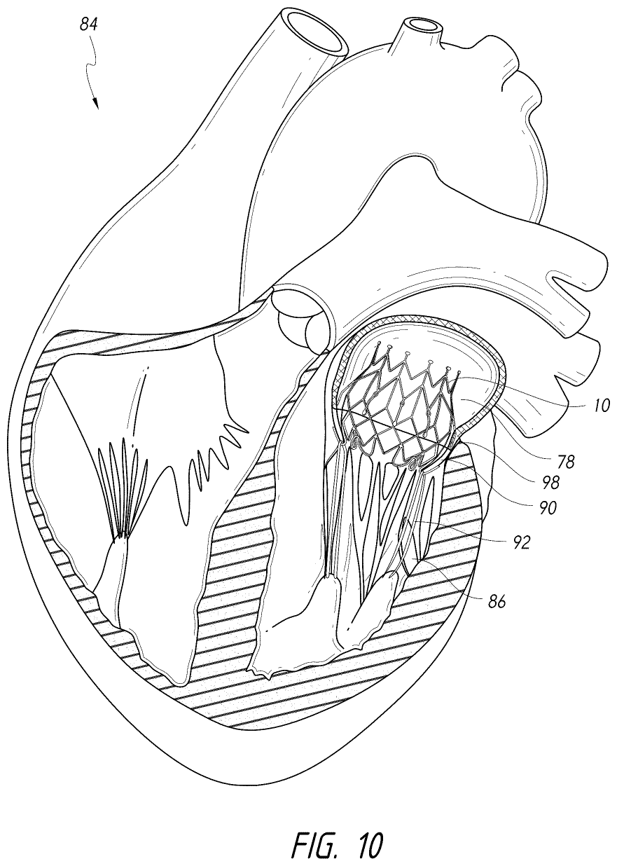

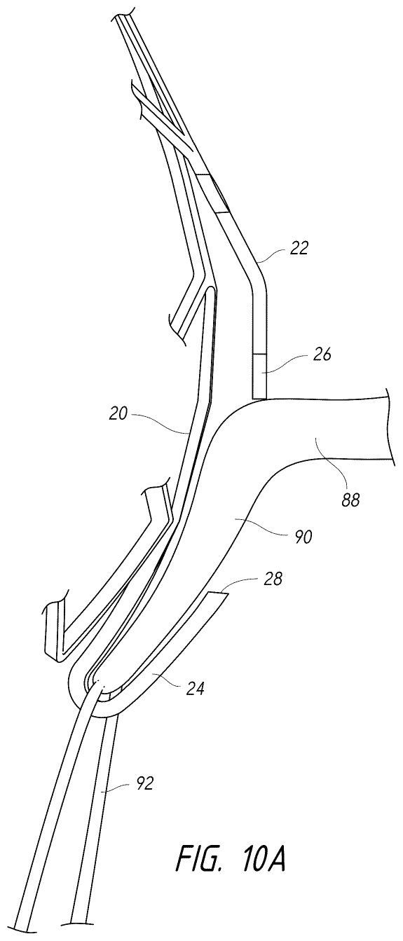

In preferred embodiments, any of the prostheses 10 described above may be deployed into a heart valve annulus, and positioned when compacted so that the anchor tips 26, 28 of the opposing anchors 22, 24 are disposed on opposite sides of the native annulus 88 as shown in FIGS. 10 and 10A. As the replacement heart valve 10 is expanded, the opposing anchors are drawn closer together so as to grasp tissue on opposite sides of the native annulus 88 and securely hold the replacement heart valve 10 in position. As such, the replacement heart valve 10 can be held securely in position without requiring a substantial radial force against the native annulus. Because the anchor tips are preferably atraumatic, the grasping or engaging of tissue by the prosthesis minimizes damage to the native tissue. The foreshortening portion 18 can be used to move the anchor tips 26, 28 closer together as the replacement heart valve 10 moves to the expanded position to thereby engage the native valve annulus. The prosthesis can be deployed into a heart valve or otherwise deployed in manners similar to those described with respect to a replacement heart valve in U.S. Publication No. 2010/0298931 and 2012/0078353 the entireties of each of which are hereby incorporated by reference and made a part of this specification. This is inclusive of the entire disclosure and is not in any way limited to the disclosure related to deployment of a replacement heart valve.

FIGS. 10 and 10A show a schematic representation of the replacement heart valve 10 installed in a human heart 84. The heart is shown in cross-section, and represents typical anatomy, including a left atrium 78 and left ventricle 86. The left atrium 78 and left ventricle 86 communicate with one another through a mitral annulus 88. Also shown schematically is a native anterior mitral leaflet 90 having chordae tendineae 92 that connect a downstream end of the anterior mitral leaflet 90 and to the left ventricle 86.

In one preferred embodiment, a method is provided of delivering a replacement valve to a native mitral valve and atraumatically securing the replacement valve relative to the native mitral valve annulus 88. The replacement valve can be mounted on a delivery device and delivered to the native mitral valve annulus while the replacement valve is in a radially compacted state. The replacement valve may be positioned so that the ends or tips of the distal anchors are on a ventricular side of the native leaflets 90 beyond a location where chordae tendineae 92 connect to free ends of the native leaflets. At least a portion of the replacement valve can be released from the delivery device to thereby expand the distal anchors radially outwardly. At this time the distal anchors may extend between at least some of the chordae. The distal anchors (along with the frame) can be moved toward the ventricular side of the native valve annulus with the distal anchors extending between at least some of the chordae tendineae to provide tension on the chordae tendineae. With tension provided on the chordae tendineae, the replacement valve can be further released from the delivery device to thereby expand the proximal anchors radially outwardly. The proximal anchors upon further release of the replacement valve from the delivery device can move into engagement with tissue on an atrial side of the native valve annulus, such as with the atrial side of the native valve annulus.

The method just described may utilize any of the prostheses herein described, but may be particularly suitable for the prosthesis of FIGS. 8A-8B where the ends of the distal anchors are not positioned as far out radially as the ends of the proximal anchors when the frame is expanded. Thus, the distal anchors may have a suitable length for extending between and providing tension on the chordae tendineae, but need not and may not in some embodiments engage tissue with the tips 28. Thus, in some embodiments the some or all of the distal anchors remain spaced from tissue on the ventricular side of the native valve annulus after delivery and expansion. The interaction between the distal anchors and the chordae tendineae may therefore be sufficient to secure the distal end of the prosthesis, while the engagement of the proximal anchors with tissue on the atrial side of the native valve annulus will help further secure and orient the prosthesis

As illustrated in FIGS. 10 and 10A, the distal anchors may comprise loops, such as any of the looped structures previously described. The distal anchors may also be covered with a resilient material such as described above for the skirt 70 that promotes tissue growth with adjacent body tissue. Such material may also be useful to prevent paravalvular leakage. The atraumatic distal anchors may advantageously prevent snagging of the prosthesis on internal structures, such as the papillary muscles.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

Similarly, this method of disclosure, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

What is claimed is:

1. A self-expanding replacement valve prosthesis comprising:
an expandable frame comprising a proximal portion and a distal portion, the expandable frame configured to radially self-expand for deployment within a native heart valve;
a valve body attached to the expandable frame, the valve body comprising a plurality of prosthetic heart valve leaflets;
a plurality of struts extending from a proximal end of the proximal portion of the expandable frame in a proximal direction;
a plurality of distal anchors extending from a base of the distal portion of the expandable frame and, when the expandable frame is in an expanded configuration, extending radially outwardly before extending generally proximally towards the proximal portion of the expandable frame, wherein each of the plurality of distal anchors is connected to the expandable frame at the base with a single strut which splits into two struts which connect at an end of the struts to form an atraumatic looped end, and wherein the plurality of distal anchors have a length sufficient to extend between and provide tension on chordae tendineae connected to native leaflets of the native heart valve;
a plurality of proximal anchors extending from the proximal portion of the expandable frame and extending radially outwardly before extending generally distally towards the distal portion of the expandable frame, wherein, when the expandable frame is in the expanded configuration, opposing anchors of the plurality of distal anchors and the plurality of proximal anchors are drawn closer together so as to grasp tissue on opposite sides of the native heart valve and wherein the plurality of distal anchors extend radially outward further than the plurality of proximal anchors; and
a first fabric portion covering at least the distal portion of the expandable frame.

2. The prosthesis of claim 1, wherein the expandable frame comprises a plurality of cells formed by interconnected struts.

3. The prosthesis of claim 1, wherein the atraumatic looped end of each of the plurality of distal anchors comprises an elongate teardrop shape.

4. The prosthesis of claim 1, wherein the atraumatic looped end of each of the plurality of distal anchors is covered by fabric.

5. The prosthesis of claim 1, wherein the two struts form a generally triangular loop.

6. The prosthesis of claim 1, wherein the expandable frame is bulbous.

7. The prosthesis of claim 1, wherein the prosthesis is a replacement mitral valve prosthesis.

8. The prosthesis of claim 1, wherein the prosthesis is a replacement tricuspid valve prosthesis.

9. The prosthesis of claim 1, wherein the prosthesis is a replacement atrioventricular valve prosthesis.

10. A self-expanding replacement valve prosthesis comprising:
an expandable frame comprising a first portion and a second portion, the expandable frame configured to radially self-expand for deployment within a native heart valve;
a valve body attached to the expandable frame, the valve body comprising a plurality of prosthetic heart valve leaflets;
a plurality of struts extending from the first portion of the expandable frame in a direction opposite the second portion of the expandable frame;
a plurality of first anchors extending from an end of the second portion of the expandable frame and extending radially outwardly before extending generally towards the first portion of the expandable frame, when the expandable frame is in an expanded configuration;
a plurality of second anchors formed from struts connected to each other at a radially outward location to form an outwardly extending loop to facilitate engagement with an atrial annulus of the native heart valve, wherein the outwardly extending loop is radially more distant from a central longitudinal axis of the expandable frame than a radius of a first end of the first portion when the expandable frame is in the expanded configuration.

11. The prosthesis of claim 10, wherein, when the expandable frame is in the expanded condition, opposing anchors of the plurality of first anchors and the plurality of second anchors are drawn closer together so as to grasp tissue on opposite sides of the native heart valve and wherein the plurality of first anchors extend radially outward from the central longitudinal axis further than the plurality of second anchors.

12. A replacement heart valve comprising:
an expandable frame comprising an atrial end and a ventricular end and having a longitudinal axis extending therebetween, the expandable frame configured to radially expand and contract for deployment within a native valve, wherein when the expandable frame is in an expanded configuration, the expandable frame having a larger cross-sectional dimension in a middle portion of the expandable frame and a smaller cross-sectional dimension in the ventricular end of the expandable frame, the middle portion being between a proximal portion of the expandable frame and a distal portion of the expandable frame;
a plurality of generally longitudinal struts extending from the atrial end, each of the plurality of longitudinal struts comprising a tab with an enlarged portion;
a plurality of anchors positioned at the distal portion of the expandable frame via a first base and a second base, wherein at least some of the plurality of anchors are configured to engage tissue on a ventricular side of a native valve annulus;
a first fabric portion covering at least a portion of the ventricular end; and
a second fabric portion covering at least a portion of the plurality of anchors.

13. The replacement heart valve of claim 12, wherein each of the plurality of anchors comprise a first segment connected to the first base, a second segment connected to the second base, and an arcuate segment connecting the first segment and the second segment.

14. The replacement heart valve of claim 13, wherein, when the replacement heart valve is in an expanded configuration, both the first segment and the second segment comprise a bend such that a tip of each anchor extends in a proximal direction.

15. The replacement heart valve of claim 14, wherein each of the plurality of anchors include a cavity between the first segment and the second segment.

16. The replacement heart valve of claim 12, wherein the plurality of anchors and the expandable frame are configured to cooperate to grasp tissue on opposite sides of the native valve annulus.

17. The replacement heart valve of claim 12, wherein at least one of the plurality of anchors is configured to grasp a native anterior leaflet.

18. The replacement heart valve of claim 12, wherein the expandable frame comprises undulating struts.

19. The replacement heart valve of claim 18, wherein the undulating struts form a elongated teardrop shape.

20. The replacement heart valve of claim 12, wherein at least a portion of the replacement heart valve comprises a self-expanding material.
